(19) United States
(12) Patent Application Publication
(10) Pub. No.: US 2025/0332013 A1
(43) Pub. Date: Oct. 30, 2025

(54) IMPLANT DELIVERY DEVICE

(71) Applicant: Terumo Corporation, Tokyo (JP)

(72) Inventor: Vladimir Satchouk, Aliso Viejo, CA (US)

(73) Assignee: Terumo Corporation, Tokyo (JP)

(21) Appl. No.: 18/997,494

(22) PCT Filed: Jul. 24, 2023
(86) PCT No.: PCT/US2023/070867

§ 371 Date: Jan. 21, 2025

Related U.S. Application Data
(60) Provisional application No. 63/369,344, filed on Jul. 25, 2022.

(51) Int. Cl. A61F 2/966; A61F 2/95; A61L 31/02; A61M 25/09

(52) U.S. Cl. CPC A61F 2/966; A61F 2/9517; A61L 31/022; A61F 2210/0033; A61L 2400/16; A61M 2025/09083; A61M 2025/09141

(57) ABSTRACT

A delivery device may include a support structure for imparting a desired stiffness, springiness, and kink-resistance. The delivery device may include a core wire and/or one or more electrical wires which are electrically connected to a detachment mechanism. A medical device such as an implant may be attached to the delivery device by a detachment mechanism. A support structure may extend alongside or around the core wire to provide support for the delivery device, particularly when pushing a larger implant through a smaller catheter. The support structure may include a support wire alongside the core wire, a hypotube surrounding the core wire, a braid braided around the core wire, and/or a coil coiled around the core wire. The core wire may be formed from a non-memory-shape material such as stainless steel and the support structure may be formed from a memory-shape material such as Nitinol.

### RELATED APPLICATIONS

This application claims benefit of and priority to U.S. Provisional Application Ser. No. 63/369,344 filed Jul. 25, 2022 entitled Implant Delivery Device, which is hereby incorporated herein by reference in its entirety.

### BACKGROUND OF THE INVENTION

Various types of implant delivery systems have been used for delivering implants or similar devices to a target location in the body. Some of these delivery systems utilize an elongated pusher to advance the implant through a catheter or outer sheath to the target location. Some pushers may also include a detachment mechanism that is used to detach the implant once the implant has reached its intended target location. Example implants include stents, embolic coils and vascular plugs.

It can be desirable for a pusher to be relatively flexible, resistant to kinks, and provide good “pushability” for advancing an implant. These characteristics may help the pusher and the implant to navigate particularly tortuous regions of vessels while also preventing kink damage to the pusher that might otherwise cause complications to a procedure. However, it can be challenging to achieve both high flexibility, high kink resistance, and good pushability since some materials are more prone to kinking as their flexibility is increased. Hence, there is an ever-present need for some pusher designs to increase both flexibility and kink resistance without sacrificing pushability.

### SUMMARY OF THE INVENTION

The present invention is directed to a delivery system for delivering a medical device to a target location within a patient's body. For example, example embodiments of the present invention may be used to delivery, position, and deploy implantable devices such as coils, stents, flow diverters, valves, filters, vascular plugs, stentrievers/thrombectomy devices, and the like within a body cavity including, but not limited to, blood vessels, fallopian tubes, malformations such as fistula or aneurysms, heart defects, and other luminal organs.

Some embodiments of the present invention are directed to an elongated pusher that comprises both non-shape-memory structural components and shape-memory structural components that extend longitudinally along at least a portion of the pusher to provide desirable flexibility, kink resistance, and pushability. The structural components may include one or more core wires, one or more structural wires, or one or more tubular structures (e.g., solid, braided, coiled structures).

An example embodiment of the delivery system may include a core wire composed of a non-shape-memory material such as stainless steel and a support structure extending between a proximal region and a distal region of the pusher. The support structure may comprise a shape-memory material such as Nitinol.

The delivery system may further include one or more electrical wires for providing power to a detachment mechanism, such as but not limited to a heater coil.

In one example embodiment, the support structure may extend alongside, such as parallel to, the core wire.

In one example embodiment, the support structure may at least partially surround the support structure.

In one example embodiment, the support structure may comprise a hypotube. The hypotube may at least partially enclose one or more electrical wires connected to a detachment mechanism which is positioned, for example, at a distal end or region of the pusher.

In one example embodiment, the support structure may comprise a support wire. At least a portion of the support wire may be tapered. In some embodiments, at least a portion of the core wire may also be tapered.

In an example embodiment, the core wire may be composed of stainless steel and the support wire may be composed of Nitinol.

In one example embodiment, the support structure may comprise a braid. The braid may at least partially enclose one or more electrical wires connected to a detachment mechanism on or near a distal end of the pusher.

In one example embodiment, the support structure may comprise a coil. The coil may be coiled around a partial or full length of the core wire.

In another example embodiment, the present invention may comprise a core wire comprised of a non-shape-memory material, a detachment mechanism for detaching an implant, a pair of electrical wires connected to the detachment mechanism, and a support structure comprised of a shape-memory material extending between a proximal region and a distal region of the pusher.

In one example embodiment, the support structure may extend parallel to and at least partially surround both the core wire and the pair of electrical wires.

In another example embodiment, the present invention may comprise a pusher for delivering a medical device through a lumen, the pusher comprising a stiffness means composed of non-shape-memory material for imparting stiffness to the pusher and a kink-resistant means for supporting the stiffness means. The kink-resistant means may extend between a proximal region and a distal region of the pusher and may comprise a shape-memory material.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, which are not to scale, in which:

FIG. 1A is an isometric view of an implant delivery device according to an example embodiment.

FIG. 1B is a side view of an implant delivery device according to an example embodiment.

FIG. 2A is an isometric view of an implant delivery device according to an example embodiment.

FIG. 2B is a side view of an implant delivery device according to an example embodiment.

FIG. 3A is an isometric view of an implant delivery device according to an example embodiment.

FIG. 3B is a side view of an implant delivery device according to an example embodiment.

FIG. 4A is an isometric view of an implant delivery device according to an example embodiment.

FIG. 4B is a side view of an implant delivery device according to an example embodiment.

FIG. 5A is an isometric view of an implant delivery device according to an example embodiment.

FIG. 5B is a side view of an implant delivery device according to an example embodiment.

FIG. 6 is a side view of an implant delivery device and implant according to an example embodiment.

FIG. 7 is a side view of an implant delivery device and implant according to an example embodiment.

FIG. 8 is a side view of an implant delivery device and implant according to an example embodiment.

FIG. 9 is a side view of an implant delivery device and implant according to an example embodiment.

### DETAILED DESCRIPTION

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

Delivery devices such as pushers and their outer delivery catheters or sheaths are typically used to advance a deliverable medical device, such as an implant, to a target location within a patient. Delivery devices will typically be advanced through a lumen of a guiding device, such as a catheter, to deliver the medical device. It is often necessary to traverse tortuous pathways, such as tight turns, when delivering such medical devices. Thus, delivery devices generally require a requisite flexibility when being advanced through the lumen of the outer guiding device so that they do not cause damage to the patient's vessels. However, the pusher of the delivery device must also have sufficient “pushability” or strength to allow distal advancement through its outer delivery catheter/sheath without kinking. The risk of such kinking or other undesirable situations may increase with increased flexibility and/or when larger medical devices are being advanced through smaller outer guiding devices.

The present invention is generally directed to a delivery device that can be used in connection with any known medical procedure in which a medical device is delivered through a guiding device, such as for delivery of an implant through a catheter. In one example, a delivery device includes an elongated delivery catheter, an elongated pusher located within the delivery catheter, and an implant located within a distal region of the delivery catheter. The pusher may be in contact with and/or releasably attached to the implant such that a physician can advance the implant distally out of the delivery catheter and disconnect the implant from the pusher's distal end. In this example, the delivery device as a whole may be advanced through a larger guide catheter that has been previously placed within a patient. In another example, the delivery device may be advanced over a previously placed guidewire. Hence, while the term delivery device is discussed in this specification with a focus on a pusher and an implant, an outer delivery catheter or delivery sheath, among other components, may also be considered part of the delivery device.

The delivery device of the present invention may include an elongated pusher that comprises both non-shape-memory structural components and shape-memory structural components that extend longitudinally along at least a portion of the pusher to provide desirable flexibility, kink resistance, and pushability. The structural components may include one or more core wires, one or more structural wires, or one or more tubular structures (e.g., solid, braided, coiled structures).

For example, the pusher may include a core wire and a support structure for reducing the likelihood of kinking and providing a desired stiffness profile to the delivery device. The core wire may be composed of a non-shape-memory material and the support structure may be composed of a shape-memory material. The non-shape-memory material may be stainless steel and the shape-memory material may be Nitinol.

The delivery device of the present invention may be configured to deliver a medical device, such as an implant, to a target location within a patient's body. The medical device may be removably attached to a distal end of the delivery device by a detachment mechanism, with the detachment mechanism being activated upon the medical device being delivered to the target location by the delivery device to release the medical device. One or more electrical wires may be connected between a power source (such as at or near a proximal region of the delivery device) and the detachment mechanism for providing a current to the detachment mechanism (e.g., to a heater coil). A filament such as a monofilament (e.g. a polymer monofilament) may extend through the heater coil as part of the detachment mechanism, and when electrical current is applied to the heater coil for heating the detachment mechanism, the monofilament may melt and detach the medical device from the delivery device.

The support structure may extend between the proximal region and a distal region of the delivery device. The support structure may extend alongside the core wire in a parallel orientation with respect to the core wire and/or one or more electrical wires. The support structure may alternatively or additionally partially or completely surround the core wire and/or one or more electrical wires. The support structure may be equal to, greater than, or less than a length of the core wire and/or one or more electrical wires.

The support structure may comprise one or more of a support wire, a hypotube, a braid, and/or a coil. One or more portions of the support wire may be tapered, such as to match a corresponding taper of the core wire. The support wire may extend parallel with respect to the core wire and/or one or more electrical wires. The support wire may be attached to the core wire and/or one or more electrical wires, such as by welding or soldering. The hypotube, braid, or coil may enclose the core wire and/or one or more electrical wires. The support structure may be partially or entirely encapsulated in an outer jacket, such as a polymer jacket.

Specific example embodiments are described further below. However, it should be understood that any of the features from any of the embodiments can be mixed and matched with each other in any combination. Hence, the present invention should not be restricted to only these embodiments, but any broader combination thereof.

The figures illustrate example embodiments of a delivery device 100 of a delivery system for delivering a medical device such as an implant 105. The type of delivery device 100 may vary in different embodiments and thus should not be construed as limited by the example embodiments shown in the figures. In an example embodiment, the delivery device 100 may comprise a pusher configured to push a medical device such as an implant 105 out of an outer delivery catheter 115 to a target location within a body (e.g., within a vessel of the body).

The delivery device 100 may include a proximal region including a proximal tip and a distal region including a distal tip. The proximal region may be defined as including up to a half of the length of the delivery device 100 from its proximal tip towards its distal tip. The distal region may be defined as including up to a half of the length of the delivery device 100 from its distal tip towards its proximal tip. The proximal region of the delivery device 100 may include a handle, either integral with or removably attachable to the proximal region, that may be grasped and manipulated by an operator when advancing or retracting the delivery device 100.

It should be appreciated that the type of implant 105 delivered by the delivery device 100 may vary in different embodiments. Non-limiting examples of such an implant 105 include an embolic device for occluding a vessel, a stent, and a vascular plug. The present invention should not be construed as being limited for use with any particular type of medical device, and thus should be construed as covering any type of medical device capable of being delivered to a target location in a body for a wide range of medical purposes, including diagnostics and treatments.

The delivery device 100 may include two or more structural components, one of which may comprise a core wire 101 which extends along all or part of its length, such as between a proximal region and a distal region of the delivery device 100. The core wire 101 may be included to impart a desired stiffness to the delivery device 100 such that the delivery device 100 may be more likely to traverse tortuous vasculatures without failing by, e.g., kicking back. The length of the core wire 101 may vary in different embodiments and may be equal to, greater than, or less than the length of the delivery device 100.

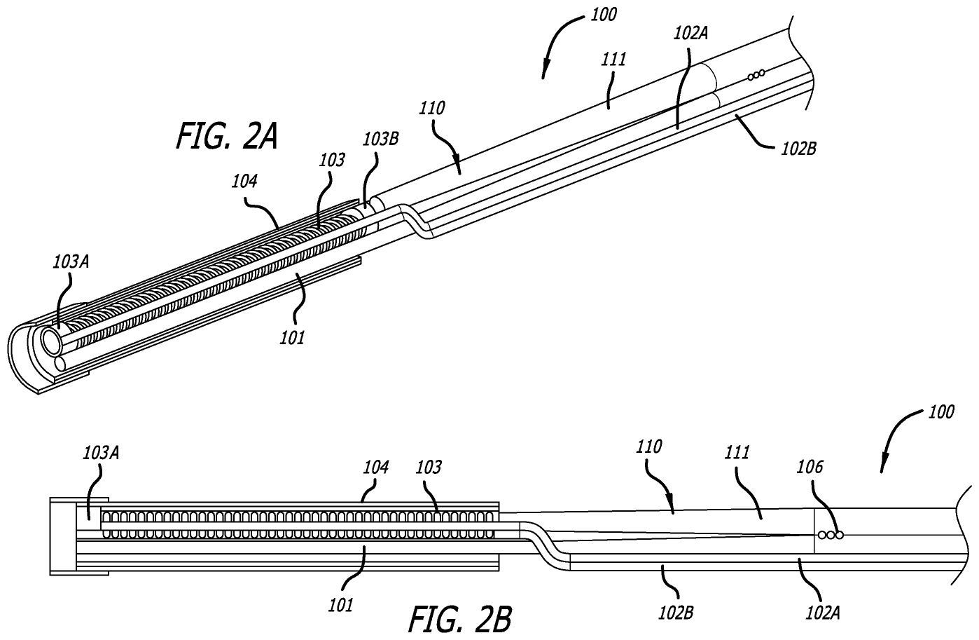

The diameter or width of the core wire 101 may also vary in different embodiments. Thus, the ratio of the diameter of width of the core wire 101 as compared to the overall diameter or width of the delivery device 100 may vary and should not be construed as limited by the example embodiments shown in the figures. In some example embodiments such as shown in FIGS. 2A-2B, at least a portion of the core wire 101 may be tapered such that the diameter of the core wire 101 decreases along at least a portion of its length. The rate and degree of tapering, such as the largest diameter or width, smallest diameter or width, and length of the taper, may vary in different embodiments.

The positioning of the core wire 101 may also vary in different embodiments. In some embodiments, the core wire 101 may be centrally located within the delivery device 100 (e.g., positioned along a central longitudinal axis of the delivery device 100). In other embodiments, the core wire 101 may be offset from a central longitudinal axis of the delivery device 100.

The type of material used for the core wire 101 may also vary in different embodiments. In one example embodiment, the core wire 101 may be composed of a non-shape-memory material such, as but not limited to, various metal or metal alloys. As a non-limiting example, the core wire 101 may be composed of stainless steel in an example embodiment.

As shown throughout the figures, one or more electrical wires 102A, 102B may extend through the delivery device 100. A proximal end of each of the electrical wires 102A, 102B may be positioned at or near a proximal region, such as at or near the proximal tip, of the delivery device 100. However, in some embodiments, the proximal end of each of the electrical wires 102A, 102B may be positioned at various other locations, including external to the delivery device 100. In some embodiments, the electrical wires 102A, 102B may be longer than the delivery device 100. In other embodiments, the electrical wires 102A, 102B may be the same length as the delivery device 100 or shorter than the delivery device 100.

Generally, the electrical wires 102A, 102B may be electrically connected to a power source. Thus, the electrical wires 102A, 102B may be utilized to power (e.g., by conducting an electrical current to) a detachment device 100 such as discussed below. The power source may be integral with the delivery device 100 or may be separately connected to the delivery device (e.g., an integral or attachable handle containing a power source).

It should be appreciated that the number of electrical wires 102A, 102B may vary in different embodiments. In some embodiments, a single electrical wire 102A may be utilized. In other embodiments, three or more electrical wires 102A, 102B may be utilized. While the present specification may occasionally refer to multiple electrical wires 102A, 102B, it should be understood that any of the embodiments shown or described herein may include only a single electrical wire 102A, or may omit electrical wires 102A, 102B entirely (e.g., when used with a mechanical detachment mechanism 103).

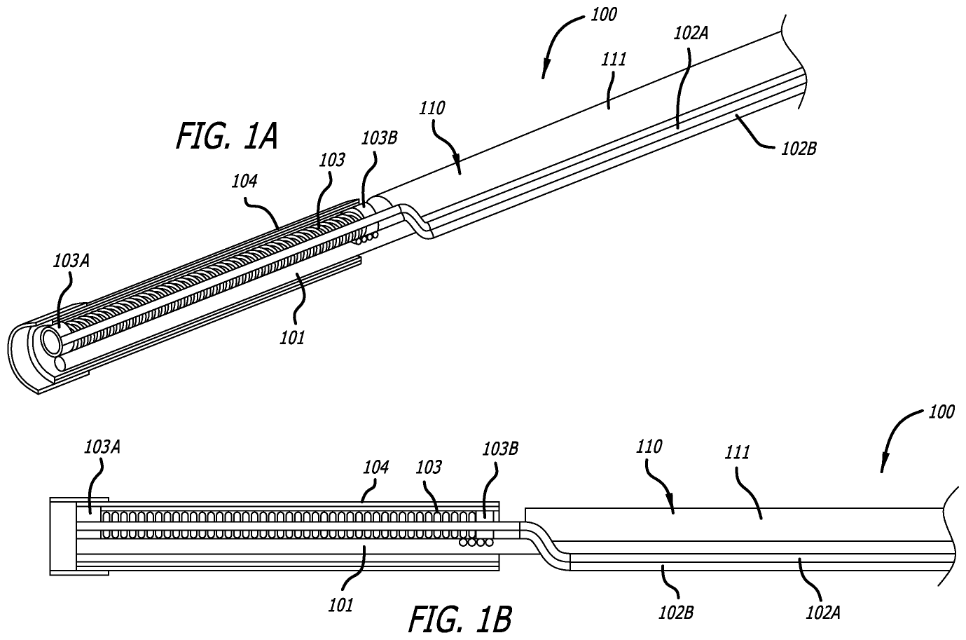

The electrical wires 102A, 102B may extend alongside the core wire 101 as shown in the figures. As shown in FIGS. 1A-5B, the electrical wires 102A, 102B may terminate at their respective distal ends into an electrode 103A, 103B. In the example embodiment shown in the figures, a first electrical wire 102A may terminate into a first electrode 103A and a second electrical wire 102B may terminate into a second electrode 103B. However, various other configurations may be utilized. For example, in embodiments having a pair of electrical wires 102A, 102B, both electrical wires 102A, 102B may terminate into a single electrode 103A.

As best shown in FIGS. 1-5B, the delivery device 100 may include or be connected to a detachment mechanism 103. The detachment mechanism 103 may be utilized to detach, at a desired time, an implant 105 or other medical device from the delivery device 100. Thus, the detachment mechanism 103 may be positioned between the delivery device 100 and an implant 105, with the detachment mechanism 103 being activated to detach the implant 105 at a target location within a body.

Various types of detachment mechanisms 103 may be utilized, including but not limited to electrically-activated detachment mechanisms 103 such as heater coils. Another non-limiting example of a detachment mechanism 103 may be comprised of various mechanical, electrical, thermal, and/or magnetic detachment mechanisms 103 known in the art. Non-limiting examples of detachment mechanisms 103 include the detachment mechanisms 103 shown and/or described in U.S. Pat. Nos. 10,980,544, 10,265,077, 9,717,500, 9,561,125, 8,460,332, 8,192,480, 8,182,506 and U.S. Publication Nos. 20060200192, 20090,062812, 20090163780, 20100268204, 20110301686, 20150289879, all of which are hereby incorporated by reference in their entireties.

In the example embodiments shown in the figures, it can be seen that the first electrical wire 102A may be connected to a distal end of the detachment mechanism 103 and that the second electrical wire 102B may be connected to a proximal end of the detachment mechanism 103. More specifically, the first electrical wire 102A may be connected to a first electrode 103A at the distal end of the detachment mechanism 103 and the second electrical wire 102B may be connected to a second electrode 103B at the proximal end of the detachment mechanism 103. It should be appreciated, however, that various other configurations may be utilized in different embodiments.

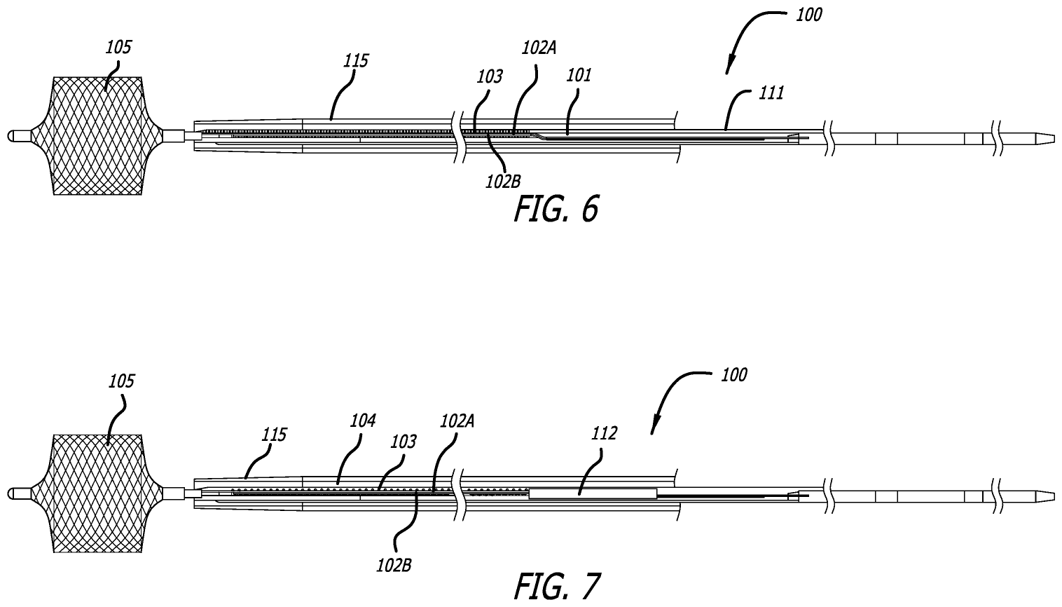

As best shown in FIGS. 6-9, a medical device, including but not limited to an implant 105 such as an occlusive device, may be removably attached to a distal end of the delivery device 100. Various types of medical devices may be delivered by the delivery device 100, including but not limited to implants 105 such as embolic devices and the like. Non-limiting examples of deliverable medical devices include the medical devices shown and/or described in U.S. Pat. Nos. 11,166,804, 11,045,205, 10,898,203, 10,729,447, 10,722,687, 10,058,330, 9,381,278, 8,603,128, 6,299,619 and U.S. Publication Nos. 20210282784, 20190046210, 20180338767, 20170189033, all of which are hereby incorporated by reference.

As shown throughout the figures, all or part of the length of the delivery device 100 may be coated in a sheath 104 or jacket which at least partially encloses or encapsulates the various internal components of the delivery device 100 discussed herein. By way of example, the sheath 104 or jacket may at least partially enclose or encapsulate one or more of the core wire 101, electrical wires 102A, 102B, detachment coil 103, and/or support structure 110. The type of sheath 104 or jacket may vary in different embodiments and may be composed of various types of materials, such as but not limited to PTFE liners or other polymers. In some embodiments, the sheath 104 may comprise a polymer jacket. The size (e.g., thickness, width, and ratio to overall size of the delivery device 100) of the sheath 104 or jacket may also vary in different embodiments.

As shown throughout the figures, the delivery device 100 may include various structural components which may impart a stiffness to the delivery device 100 to aid in kink resistance. Various embodiments may utilize different types of such structural components. Such structural components may include a combination of structural components composed of shape-memory materials and structural components composed of non-shape-memory materials. Each of the following example embodiments may include at least two such structural components; including a first structural component composed of a shape-memory material and a second structural component composed of a non-shape-memory material.

As previously mentioned, example embodiments of the present invention may include a structural component comprised of a core wire 101. The core wire 101 may be composed of a non-shape-memory material such as stainless steel or the like. The core wire 101 may extend along all or part of the length of the delivery device 100. Generally, the core wire 101 may be integrated into the delivery device 100 so as to impart a stiffness to the delivery device 100, which may offer both torque transmission and some kink resistance to the delivery device 100. However, where additional kink resistance is desired, one or more additional structural components may be included as discussed below. Such additional structural components may be composed of a shape-memory material such as Nitinol or the like.

In some embodiments, the core wire 101 may serve an additional function as an electrical conduit. In such embodiments, the core wire 101 may be composed of a conductive material so as to conduct an electrical current for various uses. By way of example, use of a core wire 101 which doubles as an electrical conduit may negate the need for one or more of the electrical wires 102A, 102B. However, in some embodiments, it should be appreciated that the core wire 101 may function as an electrical conduit in addition to, rather than instead of, one or both of the electrical wires 102A, 102B.

The use of multiple structural components, including a non-shape-memory material structural component such as a core wire 101 and a shape-memory material structural component such as various types of support structures 110 discussed below may be beneficial for improving the kink resistance of the delivery device 100. While core wires 101 do provide a modicum of kink resistance, it has been found that a core wire 101 alone may suffer from reduced effectiveness when used with larger medical devices such as implants 105 being delivered through a relatively smaller delivery device 100 such as a catheter 115.

Additionally, soldering of shape-memory materials such as Nitinol to heater coils comprised of platinum or the like can present challenges. In this respect, it is still desirable to utilize a core wire 101 composed of a non-shape-memory material such as stainless steel which is easier to solder to such heater coils. However, as mentioned above, devices having larger implants may still suffer from kinking unless additional structural components other than the core wire 101 are included. With that in mind, various example embodiments described below may include a structural component in addition to the core wire 101 to provide additional kink resistance while still maintaining the ease of soldering the stainless steel core wire 101 to the heater coil. Such additional structural components may comprise a non-shape-memory material as they do not need to be soldered to the heater coil.

Some embodiments of detachment mechanisms may require an electrical current to heat a stainless steel wire so as to melt plastic and detach various embolic devices such as implants 105. Such detachment mechanisms may require the use of stainless steel or other such conductive materials for the core wire 101 since the core wire 101 may need to be soldered to the heater coil and included in an electrical circuit. As previously discussed, the core wire 101 may thus function as an electrical conduit in some embodiments. Thus, it is not always possible to replace the core wire 101 with a shape-memory material such as Nitinol, despite such materials offering improved kink resistance. For these reasons and others, it may be desirable to include structural components in addition to a stainless steel core wire 101 as described below.

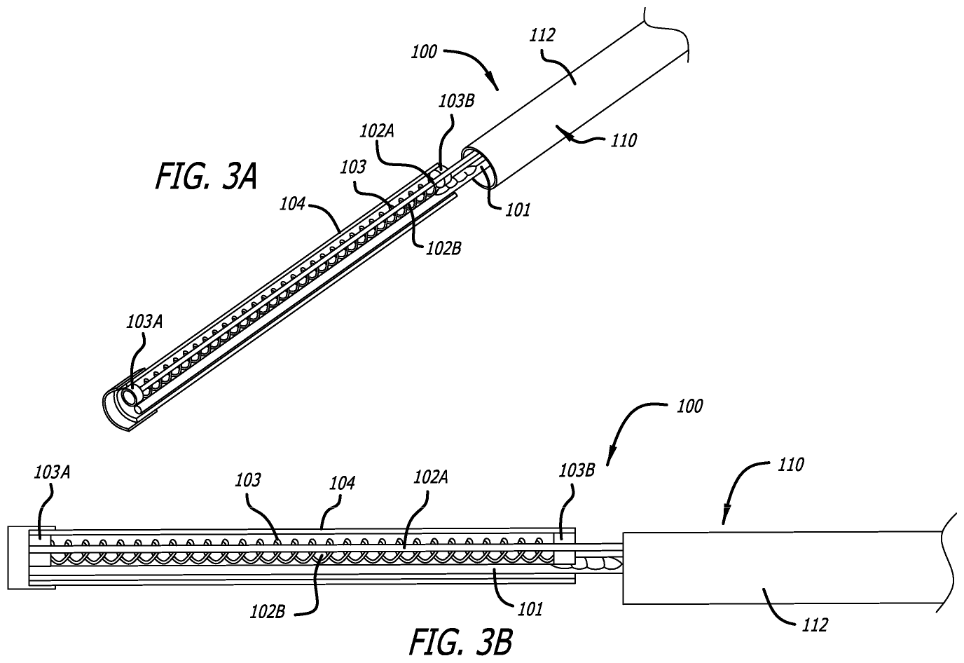

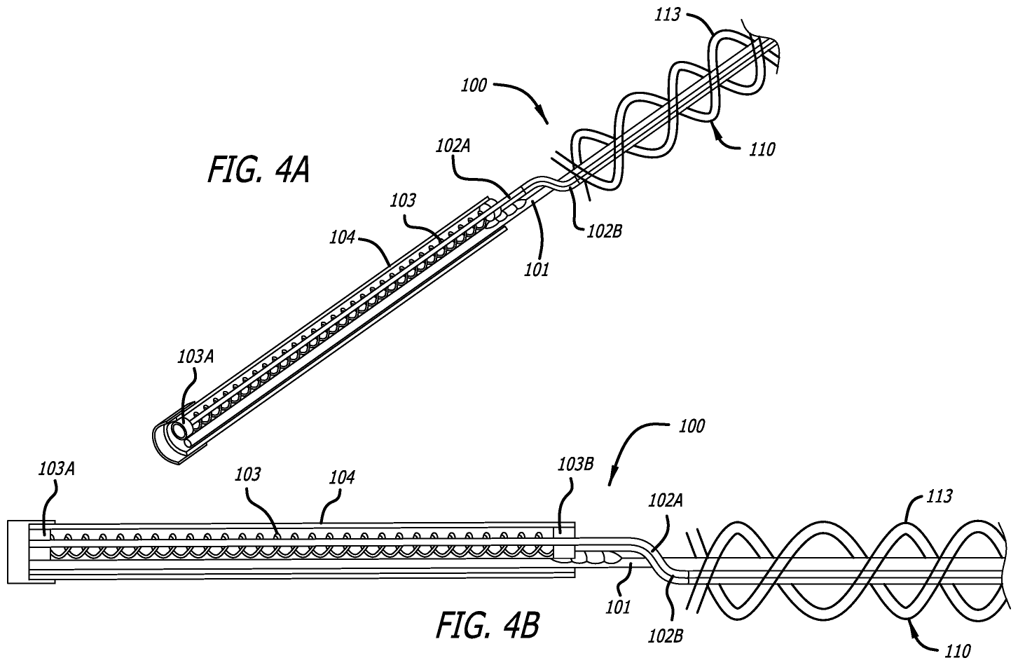

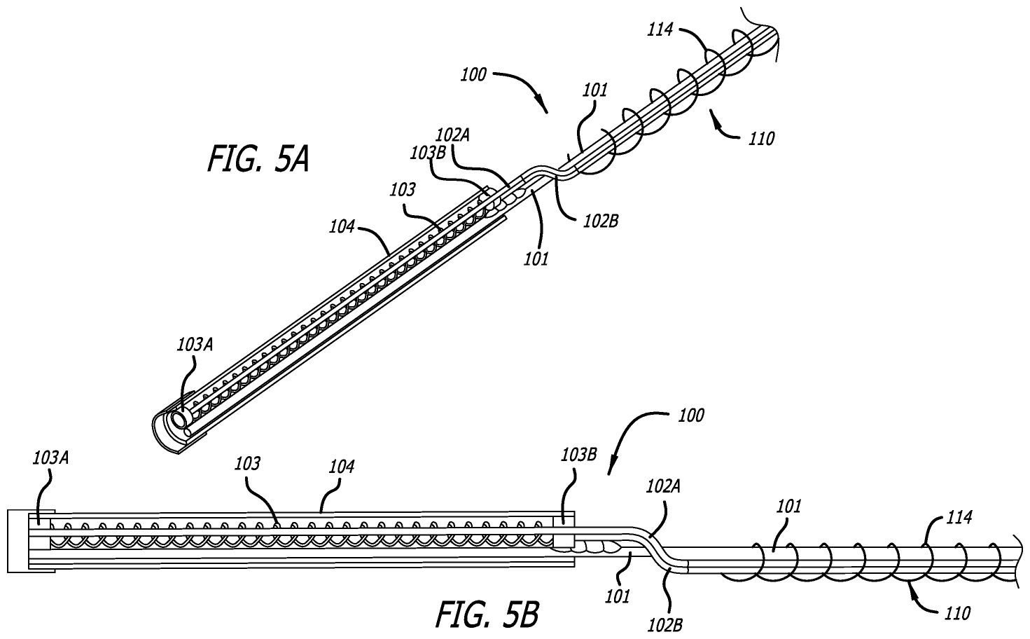

In addition to a core wire 101, the structural components of the delivery device 100 may further comprise one or more support structures 110 extending along all or part of its length. Various types of support structures 110 may be utilized. FIGS. 1A-1B illustrate an example embodiment in which the support structure 110 may be comprised of a wire 111. FIGS. 2A-2B illustrate an example embodiment in which the support structure 110 may be comprised of a tapered wire 111. FIGS. 3A-3B illustrate an example embodiment in which the support structure 110 may be comprised of a hypotube 112. FIGS. 4A-4B illustrate an example embodiment in which the support structure 110 may be comprised of a braid 113. FIGS. 5A-5B illustrate an example embodiment in which the support structure 110 may be comprised of a coil 114. Although the aforementioned example embodiments of a support structure 110 are shown and described separately herein, it should be appreciated that, in some embodiments, the delivery device 100 may include two or more of such configurations (e.g., a support wire 111 and a hypotube 112).

As shown in FIGS. 1A-5B, the support structure 110 may extend alongside, partially surround, or fully surround the core wire 101. The support structure 110 may in some embodiments be secured or attached against the core wire 101 or, in other embodiments, may not be secured or attached against the core wire 101. The manner by which the support structure 110 may be secured or attached against the core wire 101 may vary in different embodiments.

As a non-limiting example, the support structure 110 may be secured or attached against the core wire 101 by welding or soldering. FIG. 2B illustrates an embodiment showing welds 106 being used to secure a portion of the length of the support structure 110 to a portion of the length of the core wire 101. In other embodiments, in addition or alternatively to being secured or attached to the core wire 101, the support structure 110 may be wound or coiled around the core wire 101 such as shown in FIGS. 4A-5B.

It should be appreciated that the length of the support structure 110 which is attached or secured to the core wire 101 may vary in different embodiments. In some embodiments, as previously mentioned, no portion of the length of the support structure 110 may be attached or secured to the core wire 101. In other embodiments, one portion of the length of the support structure 110 may be attached or secured to one portion of the length of the core wire 101. In other embodiments, multiple, discrete (i.e., separate) portions of the length of the support structure 110 may be attached or secured to multiple, discrete portion of the length of the core wire 101. In yet other embodiments, an entire length of the support structure 110 may be attached or secured to the core wire 101.

As shown throughout the figures, the support structure 110 may extend parallel to the core wire 101. In some embodiments, all or portions of the length of the support structure 110 may extend along and be in contact with all or portions of the length of the core wire 101. In other embodiments, the support structure 110 may extend parallel to, but not in contact with, the core wire 101. In yet other embodiments as discussed herein, the support structure 110 may instead be wound or braided around the core wire 101. While not shown in the figures, some embodiments may utilize multiple configurations (e.g., a first portion of the length of the support structure 110 may extend parallel to the core wire 101 and a second portion of the length of the support structure 110 may coil around, wind around, braid around, or encapsulate the core wire 101.

In embodiments in which the support structure 110 partially or completely surrounds the core wire 101, the support structure 110 may also partially or completely surround the one or more electrical wires 102A, 102B such as shown in FIGS. 3A-5B. However, in other embodiments, the support structure 110 may partially or completely surround the core wire 101 but not any electrical wires 102A, 102B. In yet other embodiments, the support structure 110 may partially or completely surround the electrical wires 102A, 102B but not the core wire 101.

The systems and methods described herein may impart kink-resistance to a delivery device 100 such that the delivery device 100 is less prone to kinking, particularly when significant pushing force is required, e.g., for delivery of a relatively larger medical device such as an implant 105 through a relatively smaller guiding device such as a catheter 115. Thus, the various example embodiments of the delivery device 100 shown and described herein may be configured so as to traverse tortuous vasculatures, such as tight turns, without kinking.

The systems and methods described herein may also impart a desirable stiffness profile and springiness to the delivery device 100 so as to ease delivery of the medical device such as an implant 105 through the guiding device such as a catheter 115 without undue effort on the part of the operator.

FIGS. 1A and 1B illustrate an example embodiment of a support structure 110 comprised of a support wire 111. As previously discussed, the support wire 111 may comprise various materials, including but not limited to shape-memory materials such as Nitinol. In contrast, the core wire 101 may comprise a non-shape-memory material such as stainless steel or the like (although, in some embodiments, the core wire 101 may also comprise a shape-memory material such as Nitinol).

Continuing to reference FIGS. 1A and 1B, it can be seen that the support wire 111 may extend alongside and parallel to the core wire 101. The support wire 111 may extend alongside the full length of the core wire 101 (e.g., between the proximal region and distal region of the delivery device 100) or may extend alongside only a portion of the length of the core wire 101. As previously discussed, the support wire 111 may be secured or attached to the core wire 101 along its entire length, along only a single portion, or along multiple, discrete portions, such as by welding or soldering.

In the example embodiment shown in FIGS. 1A and 1B, it can be seen that the diameter or width of the support wire 111 may be greater than the diameter or width of the core wire 101. However, it should be appreciated that such an embodiment should not be construed as limiting in scope, as the diameter or width of the support wire 111 may be equal to, or less than, the diameter or width of the core wire 101 in some embodiments.

The figures illustrate an example embodiment in which the support wire 111 comprises a circular cross section. It should be appreciated, however, that the support wire 111 may have different cross sections than shown in the figures (e.g., square or triangular cross sections). The shape, size, length, and configuration of the support wire 111 may vary in different embodiments to suit different applications and to provide different stiffness profiles to the delivery device 100 overall.

FIGS. 2A and 2B illustrate an example embodiment in which a portion of both the core wire 101 and the support wire 111 is tapered (i.e., of decreasing diameter or width). In the figures, it can be seen that a portion of the length of the core wire 101 may be tapered and that a corresponding portion of the length of the support wire 111 may similarly be tapered so as to match the taper of the core wire 101. Tapering of the core wire 101 and/or support wire 111 may be desirable so as to impart a desired stiffness profile to the delivery device 100 (e.g., to prevent or reduce the chances of the delivery device 100 “kicking back” when traversing tortuous vasculatures such as tight turns).

It should be appreciated that the embodiment shown in FIGS. 2A and 2B should not be construed as limiting. In some embodiments, only one of the core wire 101 or support wire 111 may be tapered. Further, the length of the tapered portion of either of the core wire 101 or support wire 111 may vary in different embodiments. In some embodiments, an entire length of the core wire 101 and/or support wire 111 may be tapered. In other embodiments, only a single portion of the length of the core wire 101 and/or support wire 111 may be tapered. In yet other embodiments, multiple, discrete portion of the length of the core wire 101 and/or support wire 111 may be tapered.

In the example embodiment shown in FIGS. 2A and 2B, it can be seen that the taper of the support wire 111 matches the taper of the core wire 101. More specifically, it can be seen that the length of the tapered region, along with the degree of the taper, it substantially similar between the support wire 111 and the core wire 101. In other embodiments, however, the length and/or degrees of taper of the respective support wire 111 and core wire 101 may vary and not match as shown in the figures.

FIGS. 3A and 3B illustrate an example embodiment of a support structure 110 comprised of a hypotube 112. Various types of hypotubes 112 known in the art may be utilized. Generally, the hypotube 112 may comprise a cylindrical tube having an internal lumen. The core wire 101 may be positioned within the internal lumen of the hypotube 112. Additionally or alternatively, the one or more electrical wires 102A, 102B may be positioned within the internal lumen of the hypotube 112.

The length of the hypotube 112 may vary in different embodiments. The hypotube 112 may be longer than, shorter than, or equal in length with the core wire 101 and/or electrical wires 102A, 102B. In some embodiments, the hypotube 112 may extend between a proximal region and a distal region of the delivery device 100. In other embodiments, the hypotube 112 may extend for only part of the length of the delivery device 100. In an example embodiment as shown in the figures, the distal end of the hypotube 112 may terminate just prior to the proximal end of the detachment mechanism 103. In other embodiments, the hypotube 112 may extend over part or all of the length of the detachment mechanism 103.

In embodiments which use a hypotube 112 as a support structure 110 such as shown in FIGS. 3A and 3B, the electrical wires 102A, 102B and/or core wire 101 may be jacketed, such as by a liquid polymer or enameling process. In other embodiments, the electrical wires 102A, 102B and/or core wire 101 may not be jacketed but instead merely positioned within the hypotube 112 without any special treatment. In some embodiments, a heat shrink may be applied over the electrical wires 102A, 102B and/or core wire 101 prior to being inserted through the hypotube 112. In some embodiments, the hypotube 112 may function as an electrical contact itself.

The length, size, shape, and configuration of the hypotube 112 may vary in different embodiments. Generally, the hypotube 112 may be of sufficient diameter to fit the electrical wires 102A, 102B and/or core wire 101 within an internal lumen thereof. The inner diameter of the hypotube 112 may be substantially similar to an outer diameter of the electrical wires 102A, 102B and/or core wire 101, or may be greater than the outer diameter of the electrical wires 102A, 102B and/or core wire 101 such that there is extra space therein.

The hypotube 112 may be composed of various materials. By way of example, the hypotube 112 may be composed of metals, metal alloys, polymers, and the like. In one example embodiment, the hypotube 112 may be composed of a shape-memory material. In one such example embodiment, the hypotube 112 may be composed of Nitinol.

FIGS. 4A and 4B illustrate an example embodiment of a support structure 110 comprised of a braid 113. The braid 113 is generally braided around all or part of the length of the core wire 101 to provide support thereto. The braid 113 may be composed of various materials such as but not limited to metals, metal alloys, polymers/polyesters (e.g., PET or PEN), various fibers (e.g., Kevlar, fiberglass, Carbon fiber), and the like, or various combination of the preceding materials.

In one example embodiment, the braid 113 may be composed of a shape-memory material. In one such example embodiment, the braid 113 may be composed of Nitinol. The braid 113 may be composed of only a single material or, in some embodiments, may be composed of multiple materials (e.g., the braid 113 may be composed of both Nitinol and stainless steel by incorporating stainless steel into the braiding).

The configuration (e.g., braid pattern) of the braid 113 may vary in different embodiments. By way of example and without limitation, the braid 113 may be comprised of a full load braid pattern, standard braid pattern, diamond braid pattern, one-over-one (i.e., half load) braid pattern, one-over-one-under braid pattern, one-over-two-under-two braid pattern, chase wire braid pattern, tri-axe braid pattern, coil braid pattern, and the like. It should thus be appreciated that various braid patterns known in the art may be utilized for the braid 113 to impart the desired stiffness profile, among other characteristics, to the support structure 110.

It should also be appreciated that various other characteristics of the braid 113 may vary in different embodiments. For example, the number of carriers used in the braid 113 may vary. By way of example and without limitation, the braid 113 could be comprised of 4, 8, 16, 24, or 48 carriers. The braid angles and density (e.g., picks-per-inch) of the braid 113 may also vary in different embodiments. Thus, the example embodiment shown in the figures should not be construed as limiting in scope with respect to the configuration and characteristics of the illustrated braid 113.

The braid 113 will generally comprise one or more wires or strands which are wound or braided into a substantially tubular pattern. While the figures illustrate a braid 113 having a substantially circular cross section, it should be appreciated that the braid 113 could have a different shaped cross section (e.g., triangular, rectangular, or hexagonal) in some embodiments. Additionally, the width or diameter of the tube formed by the braid 113 may vary in different embodiments. Generally, the width or diameter of the tube formed by the braid 113 will be substantially the same size, or larger, than the components positioned therein (e.g., the core wire 101 and/or electrical wires 102A, 102B.

It should be appreciated that the figures herein, and in particular FIGS. 4A-4B, are not drawn to scale. Thus, the spacing, orientation, size, and number of wires forming the braid 113 should not be construed as limited by the example embodiments shown in the figures. The number of wires or strands used to form the braid 113 may vary in different embodiments. Thus, more or less wires or strands than are shown may be utilized for the braid 113. By way of example, the braid 113 may comprise any number of wires or strands, including, in some embodiments, one wire or strand or, in other embodiments, more than one wire or strand.

The braid 113 may be braided or wound around all of the length, or just part of the length, of the core wire 101. In some embodiments, the braid 113 may extend between a proximal region and a distal region of the delivery device 100. In one example embodiment such as shown in the figures, the distal end of the braid 113 may terminate at or near the proximal end of the detachment mechanism 103. In other embodiments, the braid 113 may extend over the detachment mechanism 103.

FIGS. 5A and 5B illustrate an example embodiment of a support structure 110 comprised of a coil 114. The coil 114 may itself be comprised of a coiled wire or multiple coiled wires. Generally, the coil 114 may be coiled or wound around the core wire 101 and/or electrical wires 102A, 102B such as shown in the figures.

The coil pattern may vary from what is shown in the example embodiment of the figures. Further, the number and spacing of turns of the coil 114 may vary in different embodiments. Additionally, the direction of windings (e.g., right-hand winding or left-hand winding) may also vary in different embodiments.

As best shown in FIGS. 5A and 5B, the coil 114 may be wound or coiled around the core wire 101 and/or electrical wires 102A, 102B. The coil 114 may be tight against the core wire 101 and/or electrical wires 102A, 102B, or may be loosely wound. The coil 114 may extend for all or part of the length of the core wire 101 and/or electrical wires 102A, 102B. In the example embodiment shown in the figures, it can be seen that a distal end of the coil 114 may terminate at or near a proximal end of the detachment mechanism 103. In other embodiments, the coil 114 may extend over the detachment mechanism 103.

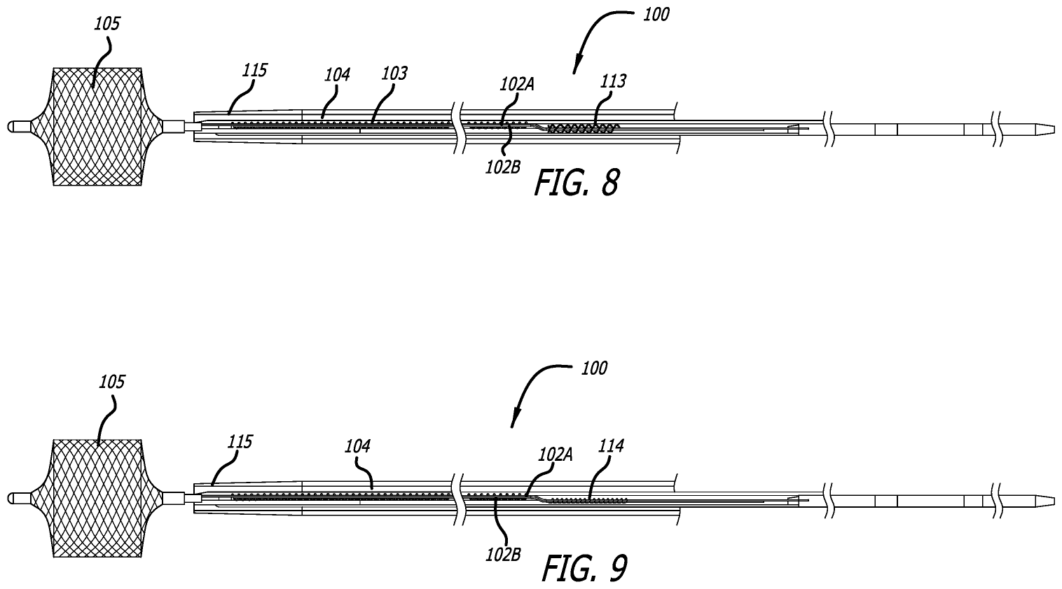

FIGS. 6-9 illustrate various embodiments of an example delivery device 100 including an attached implant 105. FIG. 6 illustrates an example embodiment of a delivery device 100 including a support structure 110 comprised of a support wire 111 including an attached implant 105. FIG. 7 illustrates an example embodiment of a delivery device 100 including a support structure 110 comprised of a hypotube 112 including an attached implant 105. FIG. 8 illustrates an example embodiment of a delivery device 100 including a support structure 110 comprised of a braid 113 including an attached implant 105. FIG. 9 illustrates an example embodiment of a delivery device 100 including a support structure 110 comprised of a coil 114 including an attached implant 105.

FIGS. 6-9 illustrate, for simplicity, an embodiment in which the support structure 110 does not extend for the full length or a majority of the length of the delivery device 100 such as a delivery catheter 115. However, it should be appreciated that the support structure 110 may extend for more of the length of the delivery device 100 than the example embodiments shown in those figures. For example, the support structure 110 may extend for substantially an entire length of the delivery device 100 in some embodiments, e.g., between a proximal region and a distal region of the delivery device 100 such as a delivery catheter 115. The support structure 110 may terminate prior to the detachment mechanism 103 or may extend alongside or over the detachment mechanism 103. The sheath 104 may extend over all, part of, or none of the length of the support structure 110.

In use, the support structure 110 will generally function to provide kink-resistance to the delivery device 100 such that the delivery device 100 is less prone to kinking when being delivered through an outer guiding device such as an introducer or catheter 115 to a target location in a patient's body. Previously, unsupported delivery devices 100 (e.g., delivery devices 100 without any of the support structures 110 described herein) have been prone to kinking during delivery; particularly when traversing tortuous vasculatures such as but not limited to the aortic arch. The systems and methods described herein utilize the support structure 110 to provide resistance to such kinking. Additionally, the use of any of the support structures 110 shown or described herein may be utilized to impart a desired stiffness profile to all or part of the length of the delivery device 100 to improve its functionality.

The systems and methods shown and described herein may be particularly suited for use in pushing larger medical devices 105 such as implants through smaller catheters 115. Generally, delivery of larger medical devices 105 such as implants requires significant pushing force, particularly if the catheter 115 through which the medical device 105 is being pushed is of a relatively smaller diameter. The use of any of the support structures 110 shown and described herein may aid in reducing the probability of kinking even when delivering larger medical devices 105 through relatively smaller catheters 115. The support structures 110 may also impart springiness to the delivery device to further reduce kinking and other undesirable responses to the delivery device 100 being pushed through a catheter 115.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1-27. (canceled)

28. A delivery device for delivering a medical device, comprising:
a core wire composed of a non-shape-memory material; and
a support structure extending at least partially between a proximal region and a distal region of the delivery device, the support structure comprising a shape-memory material; and,
wherein the support structure is comprised of a hypotube.

29. The delivery device of claim 28, wherein the support structure extends alongside and parallel to the core wire.

30. The delivery device of claim 29, wherein the core wire is at least partially surrounded by the support structure.

31. The delivery device of claim 28, wherein the non-shape-memory material is stainless steel.

32. The delivery device of claim 28, wherein at least a portion of the core wire extends through a lumen of the hypotube.

33. The delivery device of claim 32, wherein the hypotube encloses one or more electrical wires connected to a heater coil on a distal end of the delivery device.

34. The delivery device of claim 28, wherein at least a portion of the core wire is tapered.

35. The delivery device of claim 28, wherein the core wire is composed of stainless steel.

36. The delivery device of claim 35, wherein the hypotube is composed of Nitinol.

37. The delivery device of claim 28, wherein the shape-memory material is Nitinol.

38. The delivery device of claim 28, wherein the distal region of the delivery device includes a distal tip of the delivery device.

39. The delivery device of claim 28, wherein the proximal region of the delivery device includes a handle.

40. A delivery system for delivering a medical device, comprising:
a core wire, the core wire being composed of non-shape-memory material;
a detachment mechanism;
a pair of electrical wires connected to the detachment mechanism; and
a support structure extending between a proximal region and a distal region of the core wire, the support structure comprising a shape-memory material; and,
wherein the support structure is comprised of a braid.

41. The delivery system of claim 40, wherein the support structure extends alongside and parallel to the core wire and the pair of electrical wires.

42. The delivery system of claim 40, wherein the support structure at least partially surrounds the core wire and the pair of electrical wires.

43. The delivery system of claim 42, wherein the core wire is composed of stainless steel and wherein the braid is composed of Nitinol.

44. The delivery system of claim 40, wherein the braid encloses the pair of electrical wires.

45. The delivery system of claim 40, wherein the braid is wound around the core wire and the pair of electrical wires.

46. A pusher for delivering a medical device through a lumen, comprising:
a stiffness means for imparting stiffness to the pusher, the stiffness means being composed of non-shape-memory material; and
a kink-resistant means for supporting the stiffness means, the kink-resistant means extending between a proximal region and a distal region of the pusher and comprising a shape-memory material.

47. The pusher for delivering a medical device through a lumen of claim 19, wherein the kink-resistant means is comprised of a hypotube.
